(19) United States
(12) Patent Application Publication
(10) Pub. No.: US 2022/0183712 A1
(43) Pub. Date: Jun. 16, 2022

(54) Carrier Matrix for Facilitating Transfer of Skin Cores from Donor Site to Wound Site

(71) Applicants: 3D Systems, Inc., Rock Hill, SC (US); Sperry Bio, Inc., Springfield, VT (US)

(72) Inventors: Charles R. Sperry, Chester, CT (US); Ayn Lavagnino, Camas, WA (US); Vincent Piucci, East Oakham, MA (US); Karl Wassmann, Dover, MA (US); Evan Kuester, Del Mar, CA (US); John Stockwell, Sylmar, CA (US); Peter Scott Turner, Venice, CA (US); Pingyong Xu, Valencia, CA (US)

(21) Appl. No.: 17/548,727

(22) Filed: Dec. 13, 2021

Related U.S. Application Data
(60) Provisional application No. 63/124,156, filed on Dec. 11, 2020.

(51) Int. Cl. A61B 17/322

(52) U.S. Cl. CPC A61B 17/322; A61B 2017/00969; A61B 2017/3225

(57) ABSTRACT

An article is configured for transferring tissue cores from a patient donor site to a patient wound site. The article includes a matrix construction of resilient elastomeric polymer material to support an array of tissue core locators individually defining an opening and individually configured to: (1) receive a tissue core from the donor site into the opening, (2) resiliently hold the tissue core at the opening until and after the sheet is placed upon the wound site, and (3) release the tissue core when the sheet is removed from the wound site at a time that is between 2 and 29 days after the sheet is placed upon the wound site.

### CROSS-REFERENCE TO RELATED APPLICATIONS

This non-provisional patent application claims priority to U.S. Provisional Application Ser. No. 63/124,156, Entitled “Carrier Matrix for Facilitating Transfer of Skin Cores from Donor Site to Wound Site” by Charles R. Sperry et al., filed on Dec. 11, 2020, incorporated herein by reference under the benefit of U.S.C. 119(e).

### FIELD OF THE INVENTION

The present disclosure relates to skin or tissue grafting to restore a wound site. In particular, the present disclosure concerns an article and method that allow the most effective treatment of a wound site with a minimal impact upon a donor site.

### BACKGROUND

Skin grafting procedures are sometimes performed when a patient has a major wound site. Skin grafting is a surgical procedure that involves removing skin from one area of a patient's body (donor site) and transplanting it to an area of the body (recipient or wound site) where skin tissue has been damaged due to burns, injury, infection, illness, birth defects or other causes. An autograft refers to a graft in which the donor site and recipient site are on the same patient's body. One conventional approach to skin grafting is to tangentially excise a portion of skin from one part of a patient body (called a donor site) and transfer it to the wound site. This has a number of obvious drawbacks including the loss of skin from the donor site and severe scarring due to the procedure.

More recently there have been methods of grafting in which micro-cores of skin are harvested (excised) and transferred from a donor site to a recipient site. Issues with these methods include a lack of ability to maintain an evenly spaced placement and accurate orientation of the skin micro-cores until the micro-cores biologically grow into the wound bed. These issues can cause a failure of the micro-cores to form new skin or a need for an excessive harvesting. Some of these current micro-core transfer methods are tedious manual processes, leading to excessive surgical procedure lengths and surgeon hand fatigue.

### BRIEF DESCRIPTION OF THE FIGURES

FIG. 1 is a schematic diagram of an embodiment of a system for transferring skin micro-cores from a donor site to a wound site.

FIG. 2 is a flowchart of an embodiment of a process for transferring skin micro-cores from a donor site to a wound site. The carrier matrix may or may not be removed from the wound site.

FIG. 3 is a simplified isometric view of a carrier matrix.

FIG. 3A is a cross-sectional view taken through AA′ of FIG. 3 and includes a cross-section of a carrier matrix support.

FIG. 4 is an isometric drawing of a single skin core having a right circular cylinder shape with a diameter D and length L. Typical skin cores can be “generally cylindrical” but varying according to variations in an excision process and viscoelastic properties of the skin cores.

FIG. 5 is a cross-sectional view of a harvesting tool that is a component of a harvesting apparatus.

FIG. 6 is a flowchart depicting an embodiment of a first method in which a carrier matrix is used to transfer skin micro-cores from a donor site to a wound site. In this first method, the carrier matrix is placed upon the wound site for 2-29 days before being removed.

FIG. 7 is a plan view of a first embodiment of a carrier matrix having an array of skin core locators.

FIG. 7A is plan view detail taken from detail A of FIG. 7.

FIG. 8 is an isometric view of a second embodiment of a skin core locator 120.

FIG. 9 is an XY view of the skin core locator of FIG. 8.

FIG. 10 is a plan view of a third embodiment of a carrier matrix.

FIG. 10A is plan view detail taken from detail AB of FIG. 10.

FIG. 10B is isometric detail taken from detail AB of FIG. 10.

FIG. 11 is a plan view of a fourth embodiment of a carrier matrix.

FIG. 11A is plan view detail taken from detail AB of FIG. 11.

FIG. 11B is isometric view detail taken from detail AB of FIG. 11.

FIG. 12 is a plan view of a fifth embodiment of a carrier matrix.

FIG. 12A is plan view detail taken from detail AB of FIG. 12.

FIG. 12B is isometric detail taken from detail AB of FIG. 12.

FIG. 13 is a plan view of a sixth embodiment of a carrier matrix.

FIG. 13A is plan view detail taken from detail A of FIG. 13.

FIG. 14 is an isometric view of a seventh embodiment of a carrier matrix.

FIG. 14A is an isometric view taken from detail A of FIG. 14.

FIG. 15 is a flowchart depicting an embodiment of a second method in which a carrier matrix is used to transfer skin cores from a donor site to a wound site. In this second method, the carrier matrix is placed upon the wound site for less than an hour during a surgical procedure before being removed.

FIG. 16 is a flowchart depicting an embodiment of a third method in which a carrier matrix is used to transfer skin cores from a donor site to a wound site. In this third method, the carrier matrix is placed upon and then dissolves into the wound site.

FIG. 17 is a flowchart depicting an embodiment of a fourth method in which a carrier matrix is used to transfer skin cores from a donor site to a wound site. In this fourth method, the carrier matrix never contacts the wound site.

### SUMMARY

In an aspect of the disclosure, an article is configured for transferring skin cores from a patient donor site to a patient wound site. The article includes a matrix construction of resilient elastomeric polymer material to support an array of skin core locators individually defining an opening and individually configured to: (1) receive a skin core from the donor site into the opening, (2) resiliently and frictionally hold the skin core at the opening until and after the sheet is placed upon the wound site, and (3) release the skin core when the sheet is removed from the wound site at a time that is between 2 and 29 days after the sheet is placed upon the wound site.

This article has advantages that it assures both proper orientation and evenly dispersed placement of the skin cores over the array until the skin micro-cores have biologically attached to the wound bed. This is important for complete coverage and healing of the wound site while minimizing impact upon the donor site.

In one implementation, the opening is defined by an inner edge that is configured to form an interference fit with a skin core having an effective diameter in a range of 1 millimeter (mm) to 3 mm. More particularly, the opening is defined by an inner edge that is configured to form an interference fit with a skin core having an effective diameter in a range of 1 millimeter (mm) to 2 mm. Yet more particularly, the opening is defined by an inner edge that is configured to form an interference fit with a skin core having an effective diameter of about 1.5 mm. The interference fit is defined as an elastomeric deformation requirement to press or place the skin core into the opening. Skin core diameters in a range of 1 mm to 3 mm provide the least scarring of the donor site while being optimal at the wound site. A size of about 1.5 mm is the best for certain patients, donor sites, and wound sites, but other sizes within the range of 1 mm to 3 mm can be preferred for certain patients, donor sites, and wound sites.

In another implementation, the skin core locators individually include a ring and a plurality of fingers. The ring has an inner surface defining a larger inner diameter. The fingers extend inwardly from the inner surface to define a smaller inner diameter for holding a skin core having an effective diameter that is greater than the smaller inner diameter. In some variations, the skin core can have effective diameter that is about equal to the larger inner diameter. The larger inner diameter can be within a range of 1 mm to 3 mm. The larger inner diameter can be within a range of 1 mm to 2 mm. The smaller inner diameter can be in a range of 0.5 mm and 2.5 mm. The smaller inner diameter can be in a range of 0.5 mm and 1.5 mm.

In yet another implementation, the skin core locators release the skin cores because a frictional force of the skin core locator is less than a biological bonding force between skin cores and the wound site.

In a further implementation, the skin core locators release the skin cores in part due to an effect of the skin core locators absorbing bodily fluids.

In a yet further implementation, the matrix construction includes an outer frame and a lattice that couples the plurality of skin core locators to the frame.

In another implementation, the array of skin core locators have a center to center spacing that is at least twice an inner diameter of the opening.

The process disclosed herein is applicable to the translocation of epithelial, connective, nervous, and muscle tissue. “Skin” is an exemplary term used in this disclosure for clarity, but the term is applicable to all tissue types.

### DETAILED DESCRIPTION OF THE PREFERRED EMBODIMENTS

FIG. 1 is a system 2 for transferring skin micro-cores from a donor site to a wound site. In describing system 2 and in subsequent discussion, a definition of the terms wound site, donor site, and skin micro-cores is useful.

A wound site is a site of a patient's outer body to be treated. The wound site can be a site of a severe injury such as a burn, traumatic acute, chronic or surgical wound for which the site has a compromised and/or missing outer layer of skin and is to receive an equivalent of a skin graft.

A donor site is a portion of skin from which skin cores will be excised and harvested. According to the disclosure, the skin cores are removed with a generally equally spaced pattern that minimizes initial damage to the donor site and allows for a full and rapid healing of the donor site.

The skin cores are generally cylindrical or cylinder-shaped portions of skin that are excised from the donor site. The axis of an excised cylinder is along the axis or direction of cutting during excision. The cross-section of the cylinder is generally parallel to the skin at the donor site and can have any shape including circular, triangular, square, rectangular, polygonal, oval, or irregular. In an illustrative embodiment, the skin core “generally” has a shape of a right circular cylinder with a cross-section generally parallel to the skin and an axis generally along the direction of cutting or excision. In describing skin cores, the term “core” and “micro-core” can be used interchangeably.

In describing various features, the term generally refers to being by design but not necessarily exact. In other words, “generally” means by design but to within manufacturing or procedural tolerances. As for the skin core, a generally right circular cylinder shape will vary based upon cutting inaccuracy and based upon viscoelastic properties of the skin core that tend to result in distortion of the shape.

In subsequent discussions, mutually orthogonal axes X, Y, and Z will be used. With respect to the skin, the Z axis is generally perpendicular and axes X and Y generally follow the skin. When a generally cylindrical (right circular) skin core is cut, the generally circular cross-section of the skin core is generally defined along X and Y and the axis of the cylinder is defined along Z. Given that the material of the skin core is viscoelastic, the actual shape may be not be exactly a right cylinder but will be “generally” right circular cylinder as discussed earlier. Thus, these axes follow features by design but not exactly.

The system 2 according to an illustrative embodiment includes a carrier matrix manufacturing system 4, a harvesting and placement apparatus 6, and a controller 8. The carrier matrix manufacturing system 4 includes a material supply 10, a print engine 12, a post-processing system 14, and a sterile storage system 16.

The material supply 10 contains material for fabricating a carrier matrix (sheet). The material is generally polymeric in nature, but may include other components such as inorganic fillers, photoinitiators, colorants, and other components dependent upon a process performed by the print engine 12 as well as desired material properties of the carrier matrix.

The print engine 12 is configured to receive material from the material supply 10 and to fabricate the carrier matrix. In some embodiments print engine 12 is a “three-dimensional (3D) printer” can utilize photocurable resins. Alternatively, the print engine 12 can operate with non-3D printing methods such as injection molding, casting, or laser machining to name some examples. Yet alternatively, the print engine 12 can operate with hybrid methods that combine 3D print methods and non-3D print methods. For example, the print engine 12 may fuse metal or polymer particles to form a mold and then cast the carrier matrix with the mold.

In a first illustrative embodiment, the print engine 12 is based upon stereolithography and utilizes a photocurable resin. The photocurable resin is held within a resin vessel. A support within the photocurable resin defines a support surface. The print engine includes a light engine such as a laser which emits one or more of blue, violet, and ultraviolet light. In an alternative embodiment, the light engine can be a combination of a light source and a spatial light modulator. The controller is configured to operate the print engine as follows: (1) A thin layer of the photocurable resin is dispensed or formed over the support surface. (2) The light engine selectively irradiates and hardens the thin layer of photocurable resin over the support surface. (3) The support surface is repositioned and the process moves back to step (1). Steps (1)-(3) are repeated until the carrier matrix is fabricated.

In a second illustrative embodiment, the print engine is based upon a support surface, a piezo-inkjet printhead, and a blue, violet, and/or ultraviolet curing unit. The controller is configured to operate the print engine as follows: (1) The printhead selectively deposits a layer of photocurable resin above the support surface. The printhead can also deposit non-curable support material around the photocurable material. (2) The curing unit is operates to perform a “blanket” cure of all the selectively deposited photocurable material. (3) The support surface can be repositioned to receive more photocurable and support material. Steps (1)-(3) are repeated until the carrier matrix is fabricated.

The first and second illustrative embodiments are but two examples of 3D printer-based systems forming the print engine 12. Other systems can be used that form structures based upon sintering, melting, power and binder material formation, and other methods.

After the carrier matrix is formed, a post-processing system 14 can be used to clean, inspect, further cure, and/or sterilize the carrier matrix. The carrier matrix can then be transferred to a sterile storage 16. This can include individually sealing the carrier matrix into a sterile container to be stored until it is used by the harvesting and placement apparatus 6.

The harvesting and placement apparatus 6 includes a harvesting apparatus 18 and a carrier matrix support 20. The harvesting apparatus 18 is configured to excise a skin core from a donor site and to transfer the skin core to a carrier matrix which is supported by the carrier matrix support 20.

The controller 8 includes a processor 22 coupled to non-transient storage device 24. The non-transient storage device 24 stores software instructions. When executed by the processor 22, the software instructions control components of the carrier matrix manufacturing system 4 and the harvesting and placement apparatus 6. Thus, the controller 8 is configured to control components of the carrier matrix manufacturing system 4 and the harvesting and placement apparatus 6. Controller 8 can be a single physical controller or it can include a plurality of different controllers including controllers that are both internal and external to the components of the carrier matrix manufacturing system 4 and the harvesting and placement apparatus 6. In some embodiments, the controller 8 can include one or more server computers. In other embodiments, the controller 8 can include a client device such as a laptop computer, a desktop computer, a smartphone, a tablet computer, or a mobile device. When controller 8 includes multiple controllers, the controllers can operate in an integrated interconnected manner or can operate independently. As such with the variations discussed supra, the controller 8 can perform methods such as those discussed infra. That said, some specific steps of the methods of manufacturing and treatment discussed infra can be performed manually.

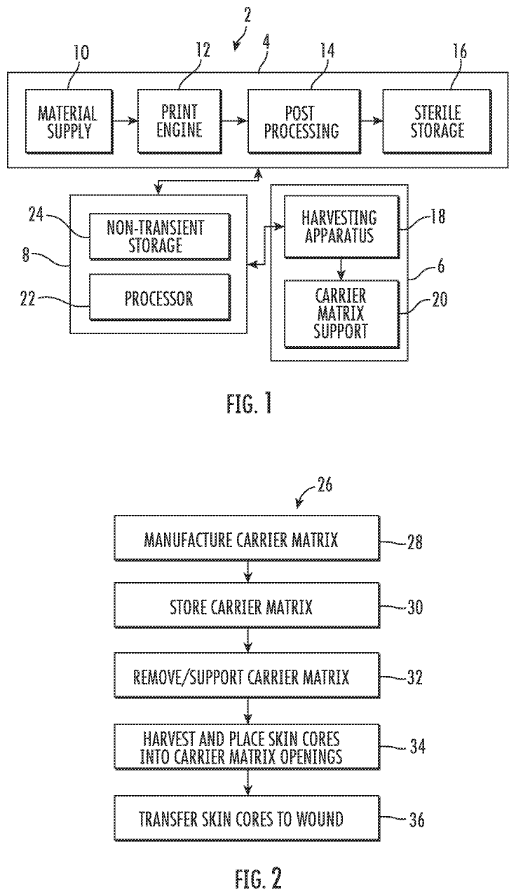

FIG. 2 is a flowchart depicting an embodiment of a method 26 of harvesting skin cores from a donor site and then transferring the harvested skin cores to a wound site. Controller 8 is configured to operate components of the carrier matrix manufacturing system 4 to perform steps 28 and 30. In step 28 the carrier matrix is manufactured by elements 10, 12, and 14 of the carrier matrix manufacturing system 4. In step 30, the manufactured carrier matrix is placed in a sterile storage 16. Between steps 30 and 32 is a storage time that can vary.

The harvesting and placement apparatus 6 can be utilized to perform steps 32-36. Controller 8 is configured to perform at least part of steps 32-36. Additional controllers may be integrated into the Harvesting Apparatus 18 to drive steps 34-36. In step 32, the carrier matrix is removed from sterile storage 16 and placed into a carrier matrix support apparatus 20. According to 34, the harvesting apparatus 18 is operated to excise skin cores from a donor site and then place the skin cores into openings that are defined by the carrier matrix. According to 36, the skin cores are transferred to the wound site. This can be performed by placing the carrier matrix onto the wound site or by transferring the skin cores from the carrier matrix to the wound site. Various alternative scenarios will be discussed infra.

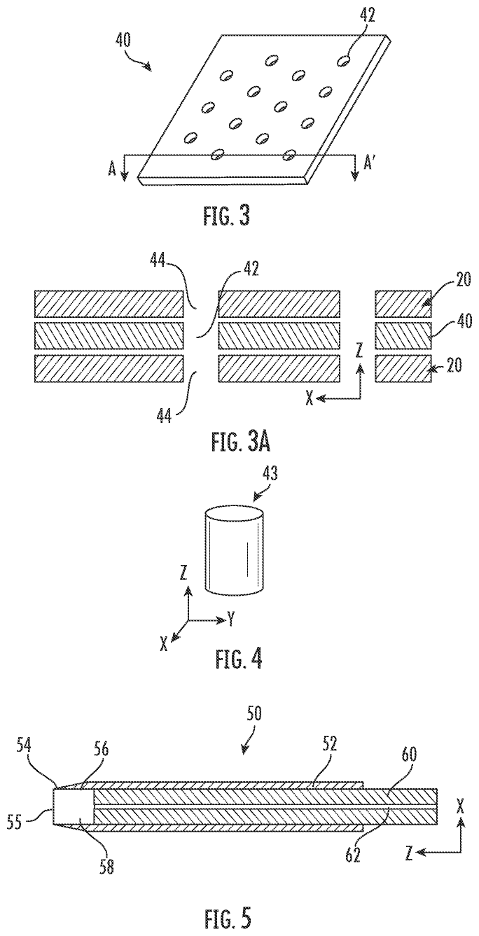

FIG. 3 is a simplified isometric view of a carrier matrix 40 that is fabricated by the print engine 12. The carrier matrix 40 is shown as a flat sheet with openings 42 defined. While illustrated in FIG. 2 as a flat sheet, a carrier matrix 40 will typically have structural features to enhance both support and release of the skin cores. Various alternative designs of carrier matrix 40 will be discussed infra.

The carrier matrix 40 is formed from a resilient and elastomeric polymeric material. The openings have a minimum diameter that is less than a diameter of a skin core. Thus, when a skin core is placed into one of the openings, an interference fit between the skin core and the material of the carrier matrix 40 compressibly and frictionally holds the skin core at the opening 42. The skin cores can individually have an outer diameter in a range of 1 to 3 millimeters (mm). For some applications, an optimal outer diameter can be in a range of 1 to 2 mm or about 1.5 mm. The inner diameter of the openings 42 can have a diameter that is between 0.1 to 1 mm less than the diameter of the skin cores, thus providing a 0.1 to 1 mm interference but perhaps more like 0.5 to 1 mm. Resilience of the skin cores and the polymeric material of the carrier matrix 40 may require this interference.

As illustrated the openings 42 have a regular spacing with a constant center to center distance. The spacing reduces a number of skin cores required to cover a given wound area thus reducing an impact on the donor site. In the illustrated embodiment, the spacing is about equal to 2 to 4 times a diameter of a skin core.

FIG. 4 is an isometric drawing depicting a single skin core 43 having a right circular cylinder shape. The skin core 43 has a diameter D defined in X and Y and a length L defined axially along Z. As discussed earlier, this is an idealized illustration of a generally cylindrical geometry that can vary based upon imperfections in harvesting and due to viscoelastic properties.

FIG. 3A is a simplified cross-section taken through AA′ of FIG. 3 but also including a cross-section of the carrier matrix support 20. The carrier matrix support 20 includes openings 44 that correspond to and are aligned with the openings 42 of the carrier matrix 40. During step 32 of method 26, a carrier matrix 40 is loaded into a carrier matrix support 20 which in turn axially aligns openings 40 and 44. While the openings 42 and 44 are illustrated as having an equal lateral dimension or diameter, the openings may have small differences in diameter.

FIG. 5 is a cross-sectional view of a harvesting tool 50 that is a component of the harvesting apparatus 18 (of FIG. 1). The harvesting tool 50 is generally cylindrical with a circular cross section defined along X and Y and a cylindrical axis along Z. The harvesting tool 50 includes a coring tool 52 having a circular cutting edge 54 and defining an opening 55 at the cutting edge 54. The coring tool 52 has an inner cylindrical surface 56 defining a cylindrical internal cavity 58. At least partially disposed within surface 56 is an extractor pin 60 having an axially extending opening 62 that can couple air, gas, and/or a vacuum source to cavity 58. The harvesting apparatus includes a movement mechanism (not shown) that can translate the harvesting tool and can move the extractor pin 60 within the coring tool 52. Thus, the coring tool 52 and extractor pin 60 can be moved independently and relative to each other along Z.

### First Embodiment: Carrier on Patient 2-29 Days

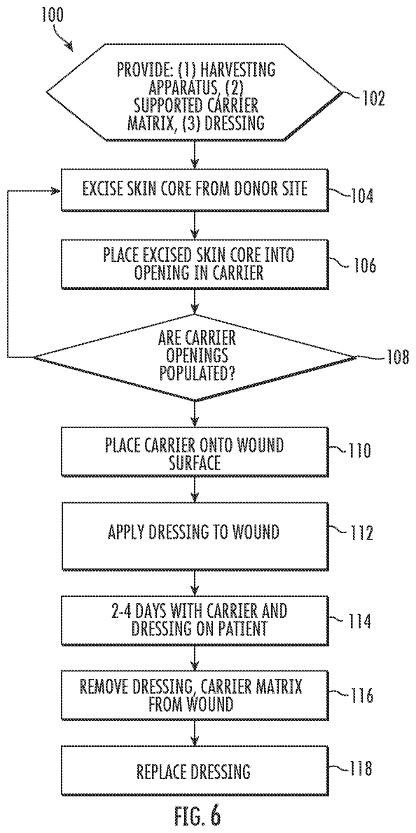

FIG. 6 is a flowchart depicting a first embodiment method 100 for treating a wound site. This method 100 begins after steps 28-32 of FIG. 2 have been performed through the operation of the carrier matrix manufacturing system 4. According to 102, the following are provided: (1) Harvesting apparatus 18, (2) carrier matrix 40 in carrier matrix support 20, and (3) a wound dressing (not shown; wound dressings are well known hospital or clinical articles for covering wounds).

According to 104, the harvesting apparatus 18 is operated to excise a skin core 43 from a donor site. In a particular embodiment, the harvesting apparatus includes the harvesting tool 50. Step 104 would have the following operations (refer to FIG. 5). (1) First, the harvesting tool is positioned with cutting edge 54 adjacent to the donor site. (2) The coring tool is displaced downward (+Z) into the skin, piercing and coring the skin so that a skin core 43 fills the cavity 58. (3) With a vacuum applied to the opening 62, the coring tool 50 is raised in the +Z direction out of the skin.

According to 106, the harvesting apparatus 18 is operated to place the excised skin core 43 into an opening 40 of the carrier matrix 40. This can be done by (1) aligning the harvesting tool 50 relative to the opening 42 (unless already aligned), (2) moving opening 55 in the +Z direction until it is proximate to the opening in Z, (3) moving the extractor pin in the +Z direction to push the skin core 43 into the opening 42, and then (4) translating the harvesting tool 50 in the −Z direction away from the carrier matrix 40. The skin core 43 is then resiliently held in the opening 40.

According to 108, steps 104 and 106 are repeated until all desired openings 42 are populated with skin cores. Then the process immediately moves to step 110 at which the carrier 40 is removed from the support 20 and is placed upon the wound site. Then, according to 112, dressing (a bandage) is applied over the wound site and carrier 40.

According to 114, a period of 2-29 days elapses, allowing the skin cores to bond to the wound site. In some embodiments, the carrier matrix 40 absorbs bodily fluids and can expand during this time. The hold of the carrier matrix 40 to the skin cores weakens due to the moisture absorption.

According to 116, the dressing and the carrier matrix 40 are removed from the wound site. The skin cores remain bound to the wound site. According to 118 the dressing is replaced.

Physical Structure of Carrier Matrix

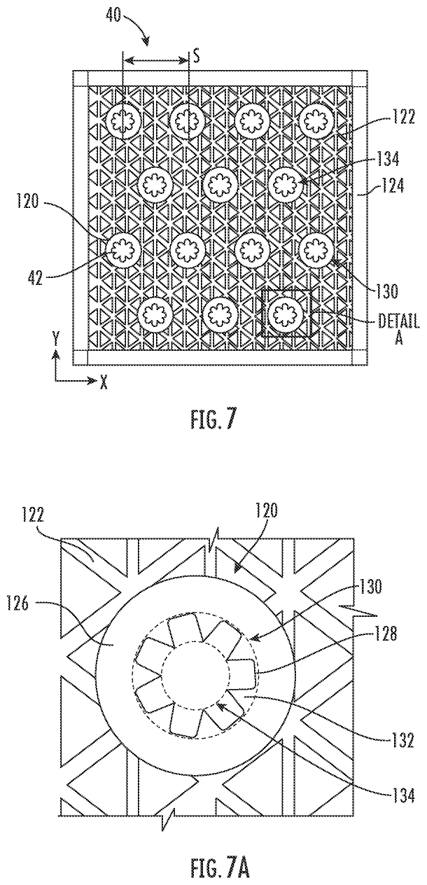

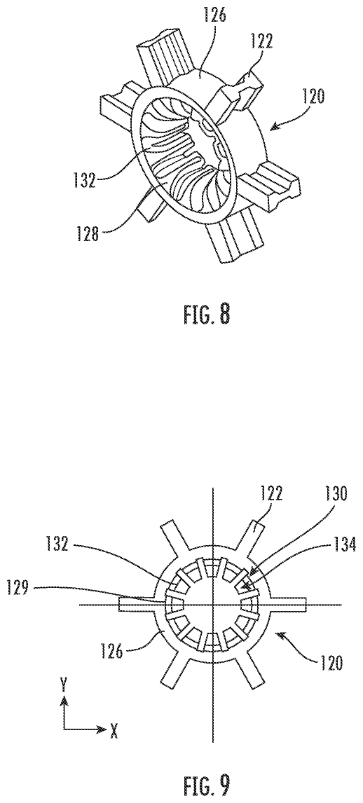

FIG. 7 is a plan view of a first embodiment of a carrier matrix 40 having an array of openings 42. The openings 42 are equally spaced in staggered rows such that an opening 42 has six nearest neighbors of equal center-to-center distance. Opening 42 is defined by a skin core locator 120. A center-to-center distance S between two skin core locators 120 is the same as the center-to-center distance S between two openings 42. A lattice structure 122 couples the skin core locators 120 to a frame 124.

FIG. 7A is “Detail A” taken from FIG. 7 with emphasis on a single skin core locator 120 supported by a portion of the lattice structure 122. The skin core locator 120 includes a ring 126 having an inner surface 128 defining a larger inner diameter 130. Extending inward from the inner surface 128 are fingers 132 that define a smaller inner diameter 134. The smaller inner diameter 134 is selected to be smaller than the diameter of the skin cores 43 to provide an interference fit between the fingers 132 and a skin core 43 being at the opening 42. The interference fit assures that the skin cores 43 will be held in a proper location and orientation within the carrier matrix 40.

FIG. 8 is an isometric view of a second embodiment of a single skin core locator 120. Fingers 132 extend inward from the inner surface 128 of ring 126 and are hollow. This serves to reduce an interference fit force between the fingers 132 and the skin core 43. 3D printing, particularly with stereolithography, enables fine and re-entrant features of the fingers 132 and the lattice 122 that may be impossible to replicate using molding or casting.

FIG. 9 is an XY view of the skin core locator 120 of FIG. 8. The inner surface 128 of ring 126 defines a larger inner diameter 130. The fingers 132 extend inwardly from the inner surface 128 and have inner or distal tips 129 that define smaller inner diameter 134.

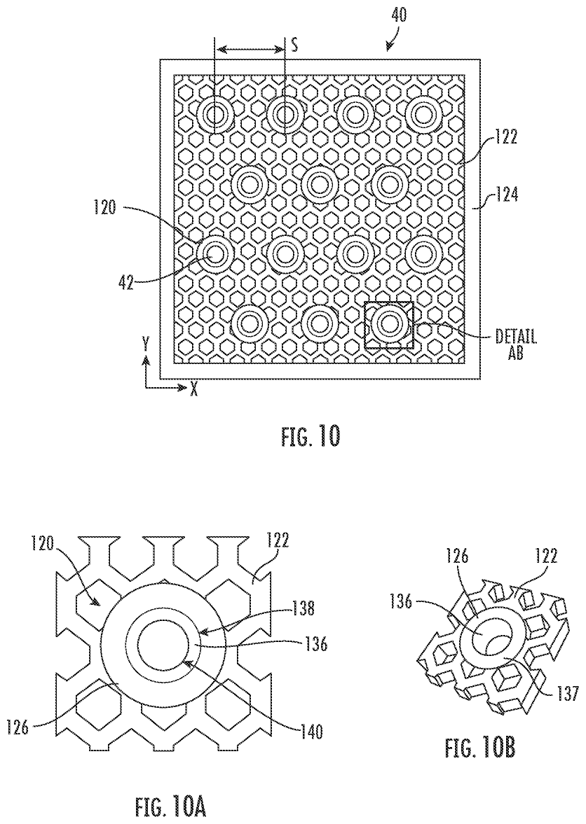

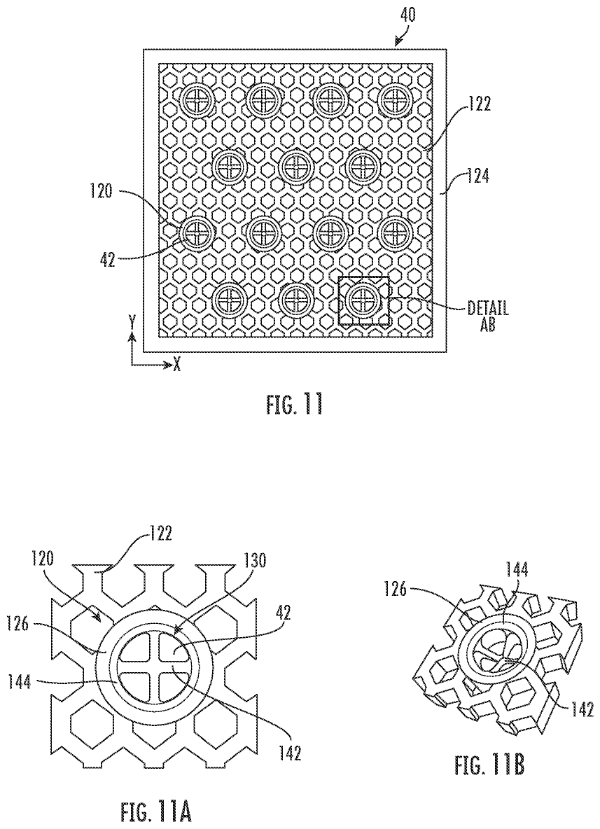

FIGS. 10-13 are various embodiments of the carrier matrix 40 that can differ from FIG. 7/7A in terms of the lattice 122 and the skin core locator 120 details. Regarding the skin core locator 120, the design of FIGS. 8 and 9 may be preferred for certain skin core 43 sizes and viscoelastic properties. However, for certain procedures, one of the designs of FIGS. 10-13 may be optimal. One advantage of the use of 3D printing is an ability to customize the geometry of the carrier matrix for a given procedure.

FIG. 10 is a plan view of a third embodiment of a carrier matrix 40 having an array of openings 42. FIG. 10A is an XY view of “Detail AB” taken from FIG. 10. FIG. 10B is an isometric view of “Detail AB” taken from FIG. 10. Comparing FIGS. 7/7A and FIGS. 10A/B, the differences are in the lattice design 122 and the skin core locator 120. In FIGS. 10A/B, the ring 126 has a conical inner surface 136 whose intersection with planar surfaces surface 137 of ring 126 defines a larger entrance diameter 138 and a smaller exit diameter 140. Surfaces 137 are parallel to the XY plane. When the skin core 43 is pressed into the opening 42 starting with the larger entrance diameter 138, it is progressively compressed as it is pressed along Z toward the smaller diameter 140. Compression of the skin core 43 against conical inner surface 136 holds it in place.

FIG. 11 is a plan view of a fourth embodiment of a carrier matrix having an array of openings 42. FIG. 11A is an XY view of “Detail AB” taken from FIG. 11. FIG. 11B is an isometric view of “Detail AB” taken from FIG. 11. Ring 126 has a curved bridging feature 142 that spans the opening 42 along two axes (X and Y in the figure) and has a concave profile along the +Z direction to hold an inserted skin core 43. Ring 126 also includes a tapered entrance surface 144 for facilitating insertion of a skin core onto the curved bridging feature 142.

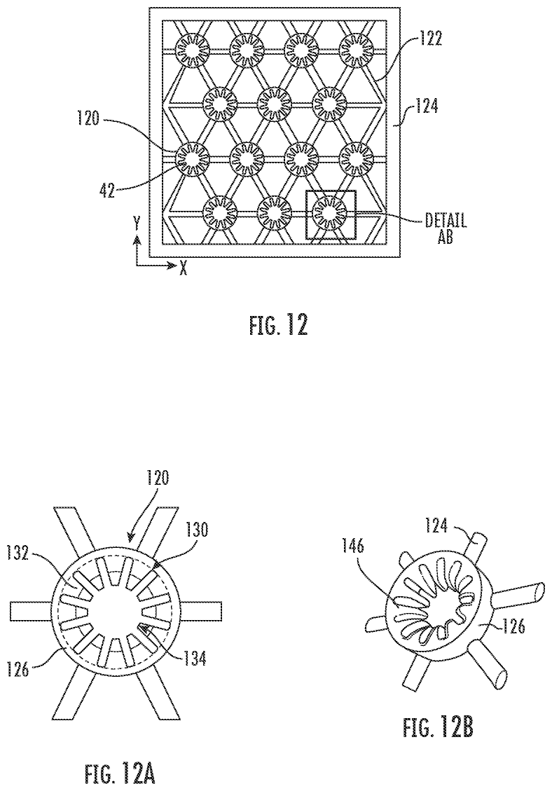

FIG. 12 is a plan view of a fifth embodiment of a carrier matrix 40 having an array of openings 42. FIG. 12A is an XY view of “Detail AB” taken from FIG. 12. FIG. 12B is an isometric view of “Detail AB” taken from FIG. 12. In the illustrated embodiment, the skin core locators 120 individually include a ring 126 with fingers 132 that curve along the +Z direction from larger inner diameter 130 to smaller inner diameter 134. More particularly, the fingers 132 have an “entrance” curved surface 146 (most visible in FIG. 12B) that facilitates insertion of a skin core 43 into opening 42.

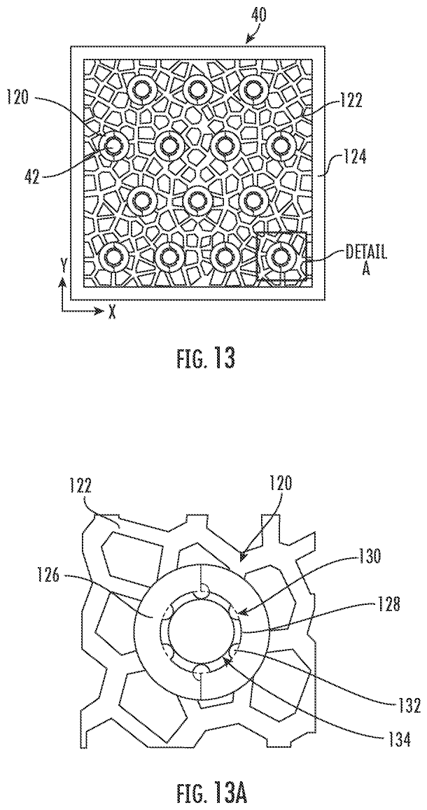

FIG. 13 is a plan view of a sixth embodiment of a carrier matrix 40 having an array of openings 42. FIG. 13A is an XY view of “Detail A” taken from FIG. 13.

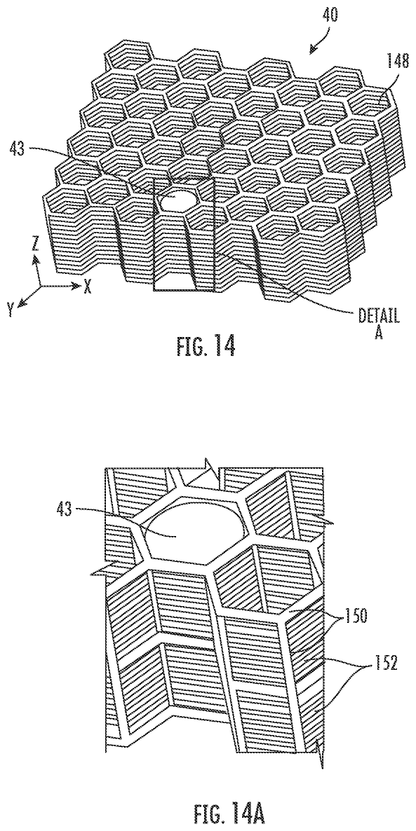

FIG. 14 is an isometric drawing depicting a seventh embodiment of a carrier matrix 40 having a “honeycomb” structure. Projected into the XY plane, the carrier matrix 40 has a hexagonal close-packed structure of cells 148. The cells 148 are individually a hexagonal cylinder with a hexagonal cross-section in X and Y and extend axially along Z.

FIG. 14A is an isometric drawing of “Detail A” taken from FIG. 14. A single skin core 43 is inserted into one of the cells 148. The cells 148 are structurally defined by main structural beams 150 and also include thinner bands 152 for frictionally holding the skin core 43 in the cell 148.

In using the embodiment of FIGS. 14/14A, a “sparse population” of skin cores 43 can be used. For example, every other cell 148 can contain a skin core 43 so that an occupied cell 148 (holding a skin core 43) is surrounded by six empty cells 148 (not holding a skin core). Other arrangements are possible such being more sparse (larger distance between skin cores) or fully populated.

Materials Used in Carrier Matrix for First and Second Embodiment

A number of different starting materials can be used for fabricating the carrier matrix depending partly upon a fabrication process employed. In an illustrative embodiment, the material is a photocurable resin with the following properties: (1) It cures rapidly with application of ultraviolet (UV) radiation, (2) it expands in a presence of a phosphate buffered saline solution (PBS) over a number of hours, (3) it is a resilient elastomeric material. The PBS is an aqueous solution with the following components:

| Component | Concentration |
|---|---|
| NaCl (sodium chloride) | 0.137 molar (M) |
| KCl (potassium chloride) | 0.0027 molar (M) |
| Na₂HPO₄ (disodium hydrogen phosphate) | 0.01 molar (M) |
| KH₂PO₄ (potassium dihydrogen phosphate) | 0.01 molar (M) |
| Water | Balance |

When “expansion” of a material is referred to it implies lateral linear expansion in PBS from a previously dry state. For example, a 25% expansion implies that a 10 millimeter linear dimension (defined in the XY plane) of a material increases to 12.5 millimeters. Also, expansion in PBS implies a similar expansion in bodily fluids, although the actual magnitude of the expansion can vary according to the specific composition of the bodily fluids.

In some embodiments, the photocurable resin contains the following component materials: (1) one or more hydrophilic monomers can be cured rapidly and provide expansion in PBS; (2) one or more hydrophobic monomers that balances properties of the hydrophilic monomers and/or provides elastic properties; (3) a photoinitiator that catalyzes a polymerization reaction with blue, violet, and/or UV radiation. The following lists examples of these component materials that can be selected and utilized at optimized concentrations.

HEAA (N-hydroxyethyl acrylamide): A water soluble monomer with a rapid cure rate. HEAA is used to define the structure of the polymer.

HBA (4-Hydroxybutyl Acrylate): A water soluble monomer with a very low glass transition temperature (Tg). This provides elasticity and a balance of hydrophilic and hydrophobic properties.

SR 9035 (Ethoxylated (15) Trimethylolpropane Triacrylate): A water soluble trifunctional monomer. This provides a rapid cure rate, flexibility, and helps control an expansion rate of the carrier in PBS.

Photomer 4050 (PEG 200 Diacrylate): A water soluble di-functional monomer. This provides a rapid cure rate and helps control expansion in PBS.

TPO-L (Ethyl (2,4,6-trimethylbenzoyl) phenylphosphinate): This is a photoinitiator; it catalyzes polymerization of the monomers.

BR7432 GB (Difunctional Aliphatic Polyester Urethane Acrylate): This is a urethane acrylate oligomer. It provides hydrophobic and elasticity properties.

ACMO (Acryloyl Morpholine): This is a water soluble monofunctional monomer having a high cure rate. Also, this provides a balanced expansion rate.

SR 217 (Cycloaliphatic Acrylate Monomer): This provides hydrophobic properties to control the expansion rate.

I-819 (Bis(2,4,6-trimethylbenzoyl)-phenylphosphineoxide): This is a photoinitiator; it catalyzes polymerization of the monomers.

### EXAMPLE FORMULATIONS

A number of different photocurable resins were individually formulated using some of the components listed supra. These includes SP-301, SP-302, and SP-303. The formulations differ primarily in terms of how much they expand in PBS.

### Formulation Example #1: ˜5% Expansion in PBS in 24 Hours

| SP-301 Component | Weight Percent |
|---|---|
| HEAA | 0.00 |
| Yellow Dye | 0.041 |
| BR7432GB | 25.500 |
| HBA | 25.500 |
| ACMO | 20.400 |
| SR 217 | 25.500 |
| I-819 | 3.060 |
| Total | 100.0 |
| Cured Material Expands ~5% in | |
| 24 Hours in PBS | |

### Formulation Example #2: ˜12% Expansion in PBS in 24 Hours

| SP-302 Component | Weight Percent |
|---|---|
| HEAA | 10.909 |
| Yellow Dye | 0.034 |
| BR7432GB | 21.559 |
| HBA | 26.104 |
| ACMO | 17.247 |
| SR 217 | 21.559 |
| I-819 | 2.587 |
| Total | 100.00 |
| Cured Material Expands ~12% in | |
| 24 Hours in PBS | |

### Formulation Example #3: 25-30% Expansion in PBS in 24 Hours

| SP-303 Component | Weight Percent |
|---|---|
| HEAA | 22.15 |
| Yellow Dye | 0.03 |
| BR7432GB | 13.29 |
| SR 9035 | 8.86 |
| HBA | 31.01 |
| ACMO | 8.86 |
| SR 217 | 13.29 |
| I-819 | 2.52 |
| Total | 100.00 |
| Cured Material Expands 25-30% in | |
| 24 Hours in PBS | |

### Second Embodiment: Carrier Removed During Procedure

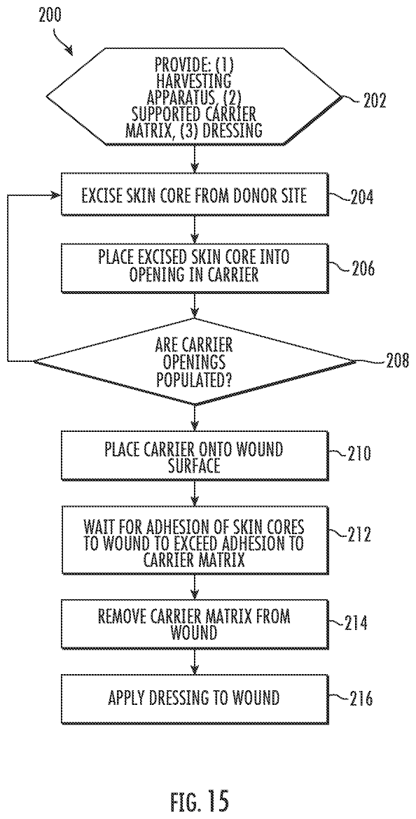

FIG. 15 is a flowchart depicting an embodiment of a second method 200 for treating a wound site. The method of FIG. 15 is similar to that of FIG. 6 except that the carrier is applied and removed during the same surgical procedure. Comparing methods 100 and 200: (1) The starting condition 102 is similar or equivalent to starting condition 202. (2) Steps 104-110 are similar or equivalent to steps 204-210 respectively.

Starting with step 212, the method diverges. Step 212 is a wait time during which the skin cores 43 gain adhesion to the wound site to exceed the adhesion to the carrier matrix 40. The wait time is typically less than one hour and is within the duration of the surgical procedure. During step 212, the carrier matrix 40 may be absorbing bodily and/or externally applied fluids to facilitate a release of adhesion between the carrier matrix 40 and the skin cores 43.

In step 214, the carrier matrix 40 is removed from the wound site. In step 216, a dressing is applied to the wound site.

Materials Used for Second Embodiment

The design and materials used for the second embodiment process 200 can have some similarities to those of the first embodiment process 100. However, the material expansion in PBS needs to be more rapid to accommodate a duration of a surgical procedure. The following is an example of a material that might be utilized for process 200:

### Formulation Example: 45-50% Expansion in PBS in 5 to 10 Minutes

| MT-103 Component | Weight Percent |
|---|---|
| HEAA | 73.96 |
| Yellow Dye | 0.19 |
| HBA | 11.41 |
| SR 9035 | 7.60 |
| Photomer 40590 (PEG200 DA) | 3.04 |
| TPO-L | 3.80 |
| Total | 100.00 |
| Cured Material Expands 45-50% in | |
| 5-10 MIN in PBS | |

### Third Embodiment: Carrier Dissolves

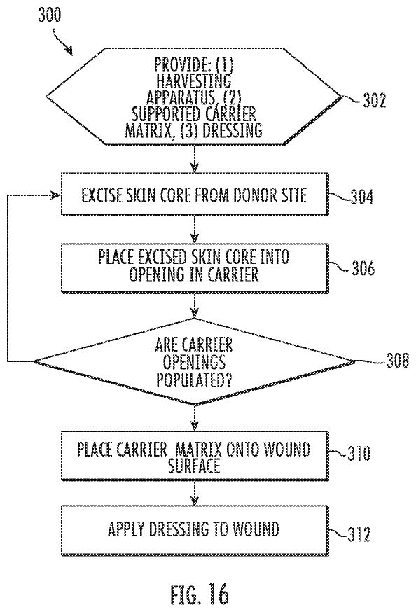

FIG. 16 is a flowchart depicting an embodiment of a method 300 for treating a wound site. The method of FIG. 16 is similar to that of FIG. 6 except that the carrier matrix 40 is not removed from the wound site. Comparing methods 100 and 300: (1) The starting condition 102 is similar or equivalent to starting condition 302. (2) Steps 104-112 are similar or equivalent to steps 304-312 respectively.

After step 312, the carrier matrix gradually dissolves into the wound site. The dressing is replaced every 2-4 days until healing is essentially complete.

### Fourth Embodiment: Carrier Never Touches Wound Site

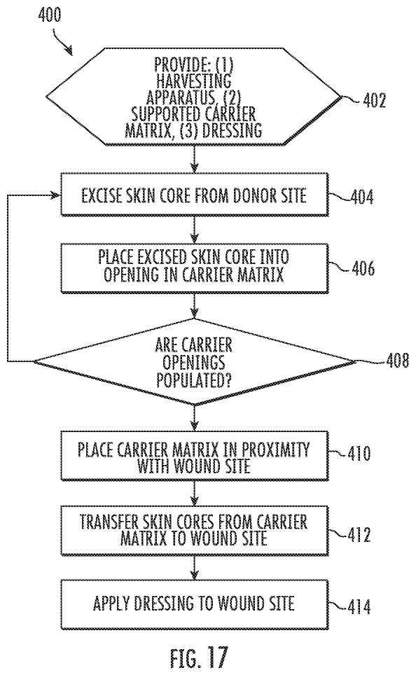

FIG. 17 is a flowchart depicting an embodiment of a method 400 treating a wound site. The method of FIG. 16 is similar to that of FIG. 15 except that the carrier matrix never contacts the wound site. Comparing methods 400 and 200:

(1) The starting condition 102 is similar or equivalent to starting condition 402.
(2) Steps 104-108 are similar or equivalent to steps 304-308 respectively.

In step 410, the populated carrier matrix 40 is placed in proximity with the wound site. In step 412, the skin cores 43 are individually transferred from the carrier matrix 40 to the wound site. According to 414, a dressing is applied to the wound site.

The specific embodiments and applications thereof described above are for illustrative purposes only and do not preclude modifications and variations encompassed by the scope of the following claims.

What is claimed:

1. An article for transferring tissue cores from a patient donor site to a patient recipient site, the article comprising:
a matrix construction of resilient elastomeric polymer material supporting an array of tissue core locators individually defining an opening and individually configured to:
receive a tissue core from the donor site into the opening;
resiliently hold the tissue core at the opening until and after the sheet is placed upon the recipient site;
release the tissue core when the sheet is removed from the recipient site at a time that is between 2 and 29 days after the sheet is placed upon the recipient site.

2. The article of claim 1 wherein the opening is defined by an inner edge that is configured to form an interference fit with a tissue core having an effective diameter in a range of 1 millimeter (mm) to 3 mm.

3. The article of claim 1 wherein the opening is defined by an inner edge that is configured to form an interference fit with a tissue core having an effective diameter in a range of 1 mm to 2 mm.

4. The article of claim 1 wherein the opening is defined by an inner edge that is configured to form an interference fit with a tissue core having an effective diameter of about 1.5 mm.

5. The article of claim 1 wherein the tissue core locators individually include:
a ring having an inner surface defining a larger inner diameter; and
a plurality of fingers extending inwardly form the inner surface to define a smaller inner diameter for holding a tissue core having an effective diameter that is greater than the smaller inner diameter.

6. The article of claim 5 wherein the tissue core has an effective diameter that is about equal to the larger inner diameter.

6. The article of claim 5 wherein the larger inner diameter is within a range of 1 mm and 3 mm.

7. The article of claim 5 wherein the larger inner diameter is in a range of 1 mm and 2 mm.

8. The article of claim 5 wherein the smaller inner diameter is in a range of 0.5 mm and 2.5 mm.

9. The article of claim 5 wherein the smaller inner diameter is in a range of 0.5 mm and 1.5 mm.

10. The article of claim 1 wherein the tissue core locators release the tissue cores because a frictional force of the tissue core locator is less than a biological bonding force between tissue cores and the wound site.

11. The article of claim 1 wherein the tissue core locators release the tissue cores in part due to an effect of the tissue core locators absorbing bodily fluids.

12. The article of claim 1 further comprising an outer frame and a lattice that couples the plurality of tissue core locators to the frame.

13. The article of claim 1 wherein the array of tissue core locators have a center to center spacing that is at least equal to twice an inner diameter of the opening.

14. The article of claim 1 wherein the resilient elastomeric polymer material expands at least five percent when exposed to an aqueous solution during a 24 hour duration.

15. The article of claim 1 wherein the resilient elastomeric polymer material expands at least ten percent when exposed to an aqueous solution during a 24 hour duration.
